(19) United States
(12) Patent Application Publication
(10) Pub. No.: US 2012/0195969 A1
(43) Pub. Date: Aug. 2, 2012

(54) TREATMENT OF ACNE BY CONDITIONED MEDIA

(75) Inventors: Neil H. Riordan, Irving, TX (US); Tierney M. Riordan, Irving, TX (US)

(73) Assignee: AIDAN RESEARCH AND CONSULTING, LLC, Chandler, AZ (US)

(21) Appl. No.: 13/249,145

(22) Filed: Sep. 29, 2011

Related U.S. Application Data
(60) Provisional application No. 61/387,885, filed on Sep. 29, 2010.

(51) Int. Cl. A61K 35/12; A61K 35/54; A61K 35/30; A61K 35/14; A61K 35/32; A61P 17/10; A61K 35/50

(52) U.S. Cl. 424/529; 424/520; 424/582; 424/583; 424/574; 424/549; 424/570

(57) ABSTRACT

Disclosed are methods and compositions for treatment of acne or acneform conditions, particularly but not limited to, acne vulgaris with products generated from culture of stem or progenitor cells. Specifically, compositions of matter are disclosed which are useful for the treatment of acne and acne associated disease states, in particular acne vulgaris, by topical administration of products derived from stem cells or progenitor cells.

### CROSS-REFERENCE TO RELATED APPLICATIONS

This application claims priority to U.S. Provisional Application Ser. No. 61/387,885, filed Sep. 29, 2010, entitled “Treatment of Acne by Conditioned Media” which is incorporated herein by reference in its entirety.

### BACKGROUND

1. Field

The current technology pertains to the field of pharmaceutical arts. More specifically, the technology relates to the treatment of acne or acneform conditions, particularly but not limited to, acne vulgaris with products generated from culture of stem or progenitor cells.

2. Description of the Related Art

Acne, the common form termed “acne vulgaris” is a prevalent skin condition that is responsible for approximately 20% of dermatologist visits and affects over 80% of teenagers. It is estimated that 17 million Americans currently suffer from acne.

The cause of common acne is an obstruction of sebaceous follicles on the face due to high levels of sebum production. This obstruction results in the formation of a microcomedo that has the possibility of progressing into a comedone. A comedone can be a whitehead or a black head or an inflammatory lesion. A blackhead occurs when the trapped sebum and bacteria (particularly propionibacterium acnes) partially open to the surface and turn black due to melanin, the skin's pigment. Blackheads can last for a long time because the contents very slowly drain to the surface.

Several methods are commonly used for treatment of this condition. Topical retinoids are generally recommended as the initial treatment of almost all new patients with acne. This is due to the fact that they are the most effective anti-comedonal agents currently available. Retinoids help disrupt acne pathogenesis by preventing the development of new microcomedones, and some possess both direct and indirect anti-inflammatory activity. Since retinoids do not possess anti-bacterial activity, the use of another agent may also be necessary to treat inflammatory activity. Retinoids enhance the follicular penetration of other agents and thus help in overall effectiveness. Other treatments include hormonal therapies or oral isotretinoin. Benzoyl peroxide, topical antibiotics such as Clindamycin, Erythromycin, Tetracyclin and oral Isotretinoin or combination of all these medications are available for mild to moderate inflammatory acne.

Numerous approaches have been described in the art for the treatment of acne. For example, U.S. Pat. No. 4,665,063 describes the use of topically applied acetyl salicylic acid, U.S. Pat. No. 4,891,227 covers the use of pads for applying anti-acne products containing salicylic acid for oily skin. Other salicylic acid-based approaches include U.S. Pat. No. 4,800,197 which covers a combination of salicylic acid and an anionic taurate surfactant, additionally, U.S. Pat. No. 5,296,476 describes the specific use of salicylic acid in combination with calcium citrate. U.S. Pat. No. 5,569,651 covers the use of a salicylic acid cream and lotions whose pH is adjusted to from about 3.8 to 4.5 using ammonium hydroxide. U.S. Pat. No. 5,871,764 discloses a salicylic acid powder formulation having a pH of from about 3 to about 4. U.S. Pat. No. 5,612,324 covers salicylic acid solutions, gels and pads having a pH of from about 2 to about 6.5. The above-cited references are incorporated by reference in their entireties.

U.S. Patent Application Publication No. 2004/0147492 discloses that tetracycline compounds, including minocycline and doxycycline, are effective in treating acne when administered to an individual in an amount that has substantially no antibiotic effect. The above-cited reference is incorporated by reference in its entirety.

### SUMMARY

The disclosure presents the unanticipated and previously undisclosed findings that stem cell conditioned tissue culture media possesses anti-acne properties. The surprising discovery includes compositions comprising mixtures of components derived from or secreted from stem or progenitor cells. The secreted components can be obtained, for example, in conditioned media. Accordingly, the present technology includes the use of said media for treatment, reduction and/or prevention of acne, as well as the use of said media for treatment, reduction and/or prevention of scarring associated with acne.

Thus, presented herein are compounds produced by stem or progenitor cells that are useful for the treatment, reduction and prevention of acne, as well as ameliorating the scarring associated with acne. Furthermore, presented herein are methods of identifying compounds from stem and progenitor cell conditioned media that can be purified and used for treatment, reduction and prevention of acne and its after-effects.

In accordance with the above, presented herein are methods and compositions for the treatment, reduction and prevention of acne and related disorders. One embodiment presented herein includes a method for the treatment of acne comprising selecting a patient in need of acne treatment; and administering to said patient one or more, or a mixture of components secreted by a stem or progenitor cell. In certain aspects, the administering may include topical administration.

Also presented herein are compositions for treatment of acne comprising: one or more, or a mixture of components secreted by a stem or progenitor cell; and a pharmaceutically-acceptable carrier or excipient. In certain aspects, the composition may be formulated in a manner to enter the pilosebaeous gland (unit), hair follicle, epidermis, dermis, or a combination thereof. In certain aspects, the composition may be formulated in a controlled release formulation, sustained release formulation, immediate release formulation, or any combination thereof. In certain aspects, the composition may be admixed with at least one anti-acne agent or medication, including those mentioned herein. In certain aspects, the anti-acne agent may include, for example, benzoyl peroxide, salicylic acid and a retinoid.

Also presented herein is a method for the reduction or prevention of acne comprising selecting a patient in need of acne prevention; and administering to said patient one or more, or a mixture of components secreted by a stem or progenitor cell.

Also presented herein is a method for the reduction or prevention of scarring associated with acne comprising selecting a patient in need of prevention of scarring associated with acne; and administering to said patient one or more, or a mixture of components secreted by a stem or progenitor cell.

Also presented herein is a method for ameliorating scarring associated with acne comprising selecting a patient in need of amelioration of scarring associated with acne; and administering to said patient one or more, or a mixture of components secreted by a stem or progenitor cell.

In certain aspects, the mixture of components secreted by a stem or progenitor cell comprises a supernatant of a cultured cell population.

In certain aspects, the supernatant can be obtained by culturing viable stem or progenitor cells under conditions that are physiological or near-physiological.

In certain aspects, the supernatant may be obtained by culturing viable stem or progenitor cells under conditions that are non-physiological.

In certain aspects, the supernatant of a cultured cell population can be substantially free of cellular debris. In some aspects, substantially free can mean that the supernatant has no more than 10%, 9%, 8%, 7%, 6%, 5%, 4%, 3%, 2%, 1%, 0.1%, 0.01%, 0.001% (wt/wt %) cellular debris.

In certain aspects, the cultured cells are exposed to conditions which may be or include, for example, one or more of a) exposure to hypoxia; b) treatment with a histone deacetylase inhibitor; c) treatment with a growth factor; d) treatment with a DNA methyltransferase inhibitor; and e) exposure to hyperthermia.

In certain aspects, the growth factor is selected from may be or include, for example, one or more of: a WNT signaling agonist, TGF-b, bFGF, IL-6, SCF, BMP-2, thrombopoietin, EPO, IGF-1, IL-11, IL-5, Flt-3/Flk-2 ligand, fibronectin, LIF, HGF, NFG, angiopoietin-like 2 and 3, G-CSF, GM-CSF, Tpo, Shh, Wnt-3a, Kirre, and a mixture thereof.

In certain aspects, the stem cell can be totipotent, capable of differentiating into cells of all histological types of the body.

In certain aspects, the at least one stem cell can be pluripotent, capable of differentiating into numerous cells of the body.

In certain aspects, the at least one stem cell can be a progenitor cell, capable of differentiating into a restricted tissue type.

In certain aspects, the totipotent stem cell may be or include, for example, one or more of an embryonic stem cell, an extra-embryonic stem cell, a cloned stem cell, and a parthenogenesis derived cell.

In certain aspects, the pluripotent stem cell may be or include, for example, one or more of a hematopoietic stem cell, an adipose stem cell, a mesenchymal stem cell, a cord blood stem cell, a placental stem cell, an exfoliated tooth derived stem cell, an endometrial regenerative cell, a hair follicle stem cell and a neural stem cell.

In certain aspects, the progenitor stem cell may be or include, for example, one or more of neuronal, hepatic, nephrogenic, adipogenic, osteoblastic, osteoclastic, alveolar, cardiac, intestinal, and endothelial progenitor cells.

In certain aspects, the embryonic stem cell can express at least one marker which may be or include, for example, one or more of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), and human telomerase reverse transcriptase (hTERT).

In certain aspects, the hematopoietic stem cell can express at least one marker which may be or include, for example, one or more of CD34, c-kit, aldehyde dehydrogenase, and the multidrug resistance transport protein (ABCG2).

In certain aspects, the adipose stem cell can express at least one marker which may be or include, for example, one or more of CD13, CD29, CD44, CD63, CD73, CD90, CD166, Aldehyde dehydrogenase (ALDH), and ABCG2.

In certain aspects, the mesenchymal stem cell can express at least one marker which may be or include, for example, one or more of STRO-1, CD73, CD90, CD105, CD54, CD106, HLA-I markers, vimentin, ASMA, collagen-1, and fibronectin, but lacking substantial expression of HLA-DR, CD117, and hemopoietic cell markers.

In certain aspects, the cord blood stem cell can express at least one marker which may be or include, for example, one or more of CD34, stem cell antigen (SCA)-1, c-kit, and CXCR-4.

In certain aspects, the placental stem cell can express at least one marker which may be or include, for example, one or more of Nanog, Oct-4, Rex-1, CD9, CD13, CD29, CD44, CD166, CD90, CD105, SH-3, SH-4, TRA-1-60, TRA-1-81, SSEA-4 and Sox-2.

In certain aspects, the exfoliated deciduous tooth stem cell can express at least one marker which may be or include, for example, one or more of STRO-1, CD146 (MUC 18), alkaline phosphatase, MEPE, and bFGF.

In certain aspects, the neural stem cell can be characterized by expression of one or more of RC-2, 3CB2, BLB, Sox-2hh, GLAST, Pax 6, nestin, Muashi-1, and prominin.

In certain aspects, the mixture of components may be administered in the form of an emulsion, gel, pack, cosmetic liquid or soap, ointments or patches.

In certain aspects, the acne is a condition which may be or include, for example, one or more of acne venenata, acne vulgaris, cystic acne, acne atrophica, acne conglobata, bromide acne, chlorine acne, acne cosmetica, acne detergicans, epidemic acne, acne estivalis, acne fulminans, halogen acne, acne indurata, iodide acne, acne keloid, acne mechanica; acne papulosa, pomade acne, premenstral acne, acne pustulosa, acne rosacea, acne scorbutica, acne scrofulosorum, acne urticata, acne varioliformis, propionic acne, acne excoriee, gram negative acne, steroid acne, or nodulocystic acne.

In certain aspects, the mixture of components further may include a thickening agent, wherein said thickening agent may be or include, for example, one or more of cetostearyl alcohol, hydrogenated lanolin, aluminum stearate, propylene glycol, and polyethylene glycol.

In certain aspects, the mixture of components further may include a chelating agent, wherein said chelating agent: (a) is present in an amount of about 0.0005% to about 1.0%: (b) is selected from the group consisting of ethylenediamine, ethylenediaminetetraacetic acid, and dimercaprol; or (c) any combination thereof.

In certain aspects, the mixture of components further can include, for example, an emulsifying agent, said emulsifying agent comprising: (a) an aqueous phase; (b) about 1% oil to about 80% oil; (c) about 0.1% organic solvent to about 50% organic solvent; (d) at least one surfactant present in an amount of about 0.001% surfactant to about 10% surfactant; (e) about 0.0005% to about 1.0% of a chelating agent; or (f) any combination thereof.

In certain aspects, the emulsifying agent can include, for example, (a) an aqueous phase; (b) about 5% oil to about 80% oil; (c) about 0.1% organic solvent to about 10% organic solvent; (d) at least one non-ionic surfactant present in an amount of about 0.1% to about 10%; (e) at least one cationic agent present in an amount of about 0.01% to about 2%; (f) about 0.0005% to about 1.0% of a chelating agent; or (g) any combination thereof.

In certain aspects, the mixture of components further may include, for example, an organic solvent.

In certain aspects, the mixture of components further can include, for example, an oil.

In certain aspects, the mixture of components further may include, for example, a silicone component, wherein said silicone compound includes at least one volatile silicone oil, wherein: (a) the volatile silicone oil can be the sole oil in the silicone component or is combined with other silicone and non-silicone oils, and wherein the other oils can be volatile or non-volatile; (b) the volatile oil used in the silicone component is different than the oil in the oil phase; (c) the silicone component may be or include, for example, one or more of methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof; or (d) any combination thereof.

In certain aspects, the mixture of components may be admixed with a volatile oil, wherein: (a) the volatile oil can be the organic solvent, or the volatile oil can be present in addition to an organic solvent; (b) the volatile oil is a terpene, monoterpene, sesquiterpene, carminative, azulene, semi-synthetic derivatives thereof, or combinations thereof; (c) the volatile oil may be or include, for example, one or more of a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives thereof, and combinations thereof; or (d) any combination thereof.

In certain aspects, the mixture of components further can include, for example, a surfactant.

In certain aspects, the mixture of components may be, for example, admixed with: (a) at least one preservative; (b) at least one a pH adjuster; (c) at least pharmaceutically acceptable buffer; or (d) any combination thereof.

In certain aspects, the mixture of components can be formulated in a manner to enter the pilosebaeous gland (unit), hair follicle, epidermis, dermis, or a combination thereof. In certain aspects, the mixture of components can be formulated in a controlled release formulation, sustained release formulation, immediate release formulation, or any combination thereof.

In certain aspects, the mixture of components can be admixed with at least one anti-acne agent. In certain aspects, the anti-acne agent may be or include, for example, one or more of benzoyl peroxide, salicylic acid and a retinoid.

In certain aspects, the mixture of components may be formulated with an excipient which may be or include, for example, one or more of carbomer 940, dimethicone, disodium lauryl sulfosuccinate, edentate disodium, glycerin, hydrated silica, methylparaben, poloxamer 182, sodium hydroxide, phosphate buffered saline, water, polymers of polyvinyl chloride, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl flouride, polyvinyl imidazole, chlorosulphonated polyolefins, and polyvinyl alcohol.

### DETAILED DESCRIPTION

The disclosure presents the unanticipated and previously undisclosed findings that stem cell conditioned tissue culture media possesses anti-acne properties. The surprising discovery includes compositions comprising mixtures of components derived from or secreted from stem or progenitor cells. The secreted components can be obtained, for example, in conditioned media. Accordingly, the present technology includes the use of said media for treatment and/or prevention of acne, as well as the use of said media for treatment and/or prevention of scarring associated with acne.

Thus, presented herein are compounds produced by stem or progenitor cells that are useful for the treatment, reduction and prevention of acne, as well as ameliorating the scarring associated with acne. Furthermore, presented herein are methods of identifying compounds from stem and progenitor cell conditioned media that can be purified and used for treatment, reduction and prevention of acne and its after-effects.

Certain embodiments of the methods presented herein include a method for the treatment, reduction of acne comprising selecting a patient in need of acne treatment; and administering to said patient one or more, or a mixture of components secreted by a stem or progenitor cell.

In some embodiments of the methods provided herein, a patient in need of acne treatment, reduction or prevention is selected. The patient in need can be any patient that potentially susceptible to one or more forms of an acne condition. The patient in need can be any patient that is currently suffering from any form of acne condition, or who has previously suffered from one or more forms of an acne condition. Acne conditions are known to those of skill in the art, and include, for example, acne venenata, acne vulgaris, cystic acne, acne atrophica, acne conglobata, bromide acne, chlorine acne, acne cosmetica, acne detergicans, epidemic acne, acne estivalis, acne fulminans, halogen acne, acne indurata, iodide acne, acne keloid, acne mechanica; acne papulosa, pomade acne, premenstral acne, acne pustulosa, acne rosacea, acne scorbutica, acne scrofulosorum, acne urticata, acne varioliformis, propionic acne, acne excoriee, gram negative acne, steroid acne, and nodulocystic acne.

### Modes of Administration and Co-Administration

The disclosed compositions may be formulated for any desirable route of delivery including, but not limited to, parenteral, intravenous, intradermal, subcutaneous, oral, topical, inhalative, transdermal, topical, transmucosal, rectal, interacisternal, intravaginal, intraperitoneal, bucal and intraocular.

In typical embodiments, the route of delivery is topical. The form of topical administration can be any suitable form that allows the components of the composition to have an anti-acne effect. For example, the composition can be administered topically in the form of an emulsion, gel, pack, cosmetic liquid or soap, ointment or patch.

It will be appreciated that in the methods provided herein, the mixture of components secreted by a stem or progenitor cell can be co-administered with one or more anti-acne agents. The anti-acne agent can be administered prior to, simultaneously, or after the administration of the mixture of components secreted by a stem or progenitor cell. For example, anti-acne agents can be administered orally while the mixture of components secreted by a stem or progenitor cell are administered topically. Any suitable orally-administered anti-acne agent can be co-administered in the methods provided herein. Representative oral anti-acne agents include, for example, antibiotics such as azithromycin, and retinoids such as retinoic acid and its derivatives (e.g., cis and trans, esters). It will be appreciated that the anti-acne agent and the mixture of components secreted by a stem or progenitor cell can both be administered topically either 1) as part of the same composition, or 2) as separate compositions either a) at the same time or b) at different times.

In embodiments where the co-administered anti-acne agent is also administered topically, the mixture of components can be admixed with at least one anti-acne agent. Any suitable topical anti-acne agent can be admixed with the mixture of components secreted by a stem or progenitor cell. Representative topical anti-acne agents include, for example, benzoyl peroxide, keratolytics, such as salicylic acid, sulfur, glycolic, pyruvic acid, resorcinol, and N-acetylcysteine; and retinoids such as retinoic acid and its derivatives (e.g., cis and trans, esters).

### Conditioned Media and Culture Conditions

In embodiments of the methods and compositions presented herein, components secreted by a stem or progenitor cell may be derived from a cultured cell population that comprises stem cells and/or progenitor cells. In some embodiments, the components secreted by the stem or progenitor cell may be obtained from the culture medium of the stem or progenitor cell. For example, in some embodiments, the components secreted by a stem or progenitor cell are derived from the supernatant of a cultured cell population. In some embodiments, the components are purified from a conditioned medium and administered in a pure or substantially pure form. In some embodiments, the components are isolated from conditioned medium, identified and then are recombinantly produced. The recombinantly produced components can then be administered to a patient in need thereof as described elsewhere herein.

The terms culture medium, culture media, growth medium, growth media and like terms refer to a solid or a liquid substance used to support the growth of cells. In reference to stem cell culture medium, the solid or liquid substance typically allows for growth of stem cells. In typical embodiments, the culture medium may be a liquid substance capable of maintaining the stem cells in an undifferentiated state. In some embodiments, however, the culture medium allows stem cells to partially or fully differentiate. The culture medium used in the methods and compositions provided herein can be, for example, a water-based medium which includes a combination of substances such as salts, nutrients, minerals, vitamins, amino acids, nucleic acids, proteins such as cytokines, growth factors and hormones, all of which are needed for cell proliferation and are capable of maintaining the stem cells in an undifferentiated state. For example, a culture medium according to this aspect of the present technology can be a synthetic tissue culture medium such as Ko-DMEM (Gibco-Invitrogen Corporation products, Grand Island, N.Y., USA), DMEM/F12 (Biological Industries, Biet Haemek, Israel), Mab ADCB medium (HyClone, Utah, USA) or DMEM/F12 (Biological Industries, Biet Haemek, Israel) supplemented with the necessary additives as is further described hereinunder. Preferably, all ingredients included in the culture medium of the present technology are substantially pure, for example, at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or at least 99.99% pure. For example, in some embodiments, the tissue culture medium comprises ingredients with a tissue culture grade.

In some embodiments, the components secreted by a stem or progenitor cell can be obtained, for example, by culturing viable stem or progenitor cells under conditions that are physiological or near-physiological. Physiological and near-physiological culture conditions can be conditions where one or more of a number of conditions mimic or approximate the range of concentrations or conditions that would occur naturally in the body of an animal. Some examples of conditions which can be adjusted to be at physiological levels include, for example, pH, temperature, CO₂ and/or O₂ concentration, glucose concentration, osmolarity, albumin, serum, cytokines, growth factors, hormones, type of feeder layer, extracellular matrix, xeno-free components, and any other condition that may affect the growth and/or differentiation of viable stem cells or progenitor cells. In certain aspects, the supernatant is obtained by culturing viable stem or progenitor cells under conditions that are non-physiological. For example, in such non-physiological conditions, one or more of a number of conditions deviate from the range of concentrations or conditions that would occur naturally in the body of an animal.

In some embodiments presented herein, stem cells can be cultured in a manner to allow viability. Any suitable culture technique which allows viability of stem cells can be used. Tissue culture solutions (media) that allow for stem cell viability include, for example, Roswell Park Memorial Institute (RPMI-1640), Dublecco's Modified Essential Media (DMEM), Eagle's Modified Essential Media (EMEM), Optimem, and Iscove's Media. The liquid medium can be supplemented with a source of serum, or alternatively, serum-free media may be used. Serum from fetal calves may typically be used, for example, at a concentration ranging from 2%-20%, more preferably at approximately 10%. In some embodiments, said fetal calf serum can be heat-inactivated, for example, by incubation at 55° C. for 1 hour, in order to neutralize complement activity. In other embodiments, human serum can be used as a substitute for fetal calf serum.

Thus, for example, culture of said stem or progenitor cells for the generation of conditioned media may be performed according to a variety of techniques known to one skilled in the art. Relevant conditions that attention must be paid to include temperature, pH, atmospheric pressure, flow rate, oxygen and carbon dioxide concentrations, as well as osmolarity of tissue culture media. For example heparin, buffers, zwitterions, or artificial/natural oxygen carriers can be added to the tissue culture. In some embodiments inhibitors of caspases may be added to maintain cell viability in culture while inhibiting apoptosis. In some aspects, said conditions can be monitored during the culture period and adjusted accordingly to achieve desired properties of the conditioned media desired.

The terms conditioned media, conditioned medium, and like terms refer to a culture medium that cells have been cultured in for a period of time. Conditioned media can be collected from cells that are originally plated at a concentration, for example, between 20-8000 cells/cm², more preferably between 2000-8000 cells/cm², and more preferably at an approximate concentration of 4000 cells/cm². For example, one of skill in the art, in view of the instant disclosure, may identify ideal concentrations of cells to be cultured based on assessment of viability, growth factor production, and generation of anti-inflammatory activity. Cells may be cultured in media for any period of time sufficient to allow secretion of components into the culture medium. For example, conditioned media may be collected at about 12, 18, 24, 30, 36, 42, 48, 54, 60, 66, 72, 78, 84, 90 or about 96 hours or more of culture. In some embodiments, the medium may be further filtered to remove cellular debris. In certain embodiments, the supernatant of a cultured cell population can be substantially free of cellular debris. It will be appreciated that in some embodiments, the supernatant can contain less than 10%, 9%, 8%, 7%, 6%, 5%, 4%, 3%, 2%, 1%, 0.5%, 0.1%, 0.05%, 0.01%, 0.005%, 0.001% or less than 0.0001% (wt/wt) cellular debris, or the supernatant can be completely free of cellular debris. Cellular debris can be removed by any suitable means including, for example, centrifugation, filtration and any other methodology which separates insoluble matter from a liquid medium.

In some embodiments, the conditioned medium can be concentrated. In some embodiments, conditioned medium is concentrated depending on the concentration of acne inhibiting compounds desired. Any suitable type of concentration can be used which allows for one or more, or a mixture of secreted components to be obtained for use in the methods and compositions presented herein. For example, molecular weight filter can be used. For example, a filter such as an Amicon 3000 Stir Cell can be used to reduce the volume and at the same time remove low molecular weight salts. In some embodiments, concentration of acne inhibiting components of the conditioned media may be achieved by column chromatography. In some embodiments, lyophilization is performed to remove the water in the medium, effectively concentrating the effective components. Concentrated conditioned media can subsequently be re-mixed with a suitable solution and administered topically.

### Identifying and Purifying Therapeutic Factors

Further embodiments include a method of optimizing therapeutic factor production from said stem or progenitor cells for acne-inhibiting properties through the use of filters that separate compositions based on electrical charge, size or ability to elute from an adsorbent. Numerous techniques are known in the art for purification of therapeutic factors and concentration of said agents. For some particular uses said stem or progenitor cell derived compounds can be sufficient for use as culture supernatants of said cells in media. Currently, media useful for this purpose can include Roswell Park Memorial Institute (RPMI-1640), Dublecco's Modified Essential Media (DMEM), Eagle's Modified Essential Media (EMEM), Optimem, and Iscove's Media.

Culture conditioned media may be concentrated by any suitable filtering/desalting techniques including use of Amicon filters with specific molecular weight cut-offs. For example, said cut-offs may select for molecular weights higher than 1 kDa to 50 kDa. Supernatant may alternatively be concentrated using any of a number of suitable concentration methodologies. For example, in some embodiments, the supernatant can be concentrated using solid phase extraction using C18 cartridges (Mini-Spe-ed C18-14%, S.P.E. Limited, Concord ON). Said cartridges are prepared by washing with methanol followed by deionized-distilled water. Up to 100 ml of stem cell or progenitor cell supernatant may be passed through each of these specific cartridges before elution, it is understood of one of skill in the art that larger cartridges may be used. After washing the cartridges material adsorbed is eluted with 3 ml methanol, evaporated under a stream of nitrogen, redissolved in a small volume of methanol, and stored at 4° C. Before testing the eluate for activity in vitro, the methanol is evaporated under nitrogen and replaced by culture medium. Said C18 cartridges are used to adsorb small hydrophobic molecules from the stem or progenitor cell culture supernatant, and allows for the elimination of salts and other polar contaminants. It may, however be desired to use other adsorption means in order to purify certain compounds from said stem or progenitor cell supernatant. Said concentrated supernatant may be assessed directly for biological activities useful for the practice of this technology, or may be further purified. Further purification may be performed using, any of a number of suitable purification techniques, such as gel filtration. As one example, gel filtration can be performed using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5×54 cm glass column and equilibrated with 3 column volumes of the same buffer. Stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically.

In order to identify and standardize stem and progenitor cell derived compounds with acne therapeutic properties, one embodiment of the technology is the concept of “units of activity” for quantification of said properties. As used herein, one Unit can be defined as the concentration of said stem or progenitor derived compounds as having sufficient activity to stimulate a biological response in an in vitro setting to a certain degree. Depending on use, this can be stimulation of a standardized cell culture to proliferate by a certain percentage, in other desired uses the Unit may designate the amount needed to inhibit differentiation a specified culture condition by a defined percentage. In a specific embodiment, one Unit can be the activity sufficient to inhibit production of the inflammatory compound TNF-alpha by 50% in a culture of 0.5 ug/ml endotoxin stimulated culture of RAW macrophage cell line cultured at a concentration of 10⁴ cells per plate in flat-bottom 96 well plates. Other methods of quantifying activity may be chosen based on other desired biological activities relevant to the pathology of acne. Without being bound to mechanism, said activities include: inhibition of inflammation; inhibition of scarring; stimulation of cellular turnover and anti-microbial activity.

### Stem Cells and Progenitor Cells

In some embodiments, stem cell or progenitor cell compounds useful for the treatment and prevention of acne can be collected from tissue culture media that has been conditioned by said stem or progenitor cells. Said stem or progenitor cells useful for the practice of the technology may be selected from a variety of cells known to one of skill in the art. Some examples of stem or progenitor cells are described further herein.

It will be appreciated that the components described herein as secreted by stem cells or progenitor cells can be collected from culture medium and identified, cloned, synthesized and/or recombinantly produced. For example, the components can be cloned into any suitable expression vector for expression from a host organism. In such embodiments, the host organism need not be a human cell, but can also be any suitable host cell or organism.

In certain embodiments stem or progenitor cells may be “activated” ex vivo by a brief culture in hypoxic conditions in order to upregulate production of therapeutic factors. Without being bound to theory, said factors may be upregulated by nuclear translocation of the HIF-1 transcription factor. Hypoxia may be achieved by culture of cells in conditions of 0.1% oxygen to 10% oxygen, preferably between 0.5% oxygen and 5% oxygen, and more preferably around 1% oxygen. Cells may be cultured for a variety of timepoints ranging from 1 hour to 72 hours or more, typically from 13 hours to 59 hours and more typically around 48 hours.

In addition to induction of hypoxia, other therapeutic properties can be endowed unto said stem or progenitor cells through treatment ex vivo with factors such as de-differentiating compounds, proliferation inducing compounds, or compounds known to endow and/or enhance stem or progenitor cells to possess properties useful for the practice of the current technology. In one embodiment cells are cultured with an inhibitor of the enzyme GSK-3 in order to enhance expansion of cells with pluripotent characteristics while not increasing the rate of differentiation. In another embodiment, cells can be cultured in the presence of a DNA methyltransferase inhibitor such as 5-azacytidine in order to endow a “de-differentiation” effect. In another embodiment, cells can be cultured in the presence of a histone deacetylase inhibitor. In other embodiments, cells can be cultured in the presence of a growth factor. In some embodiments, cells can be exposed to conditions of hyperthermia.

### Placental Tissues

In one embodiment, placental mesenchymal or mesenchymal-like stem cells may be purified directly from placental tissues, said tissues including the chorion, amnion, and villous stroma [1,2]. In another embodiment, placental tissue is mechanically degraded in a sterile manner and treated with enzymes to allow dissociation of the cells from the extracellular matrix. Such enzymes include, but not restricted to trypsin, chymotrypsin, collagenases, elastase and/or hyaluronidase.

In another aspect of the technology, media useful for treatment of acne is generated by conditioning with placental, Wharton's Jelly, or placental cord derived stem cells. Said stem cells can be of the mesenchymal lineage capable of orthodox differentiation into bone, cartilage and fat, as well as expressing prototypical mesenchymal stem cell markers CD90, CD105 and lacking substantial expression of MHC II, CD34 and CD14. Additionally said stem cells may be selected based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2.

In one embodiment, placental perivascular cells, or “human umbilical cord perivascular cells” may be used as a source of placental mesenchymal cells. Said cells may be extracted from pieces of human umbilical cord, approximately 4-5 cm long, that are dissected by first removing the epithelium of the umbilical cord section along its length to expose the underlying Warton's Jelly. Each vessel, with its surrounding Wharton's Jelly matrix, is then pulled away, after which the ends of each dissected vessel are tied together with a suture creating “loops” that are placed into a 50-mL tube containing a solution of 0.5-0.75 mg/mL collagenase (Sigma, St. Louis, Mo.) with phosphate-buffered saline (PBS, Invitrogen/Gibco, Carlsbad, Calif.). After 18 h, the loops may be removed from the suspension, which is then diluted with PBS to reduce the viscosity of the suspension and centrifuged. Following the removal of the supernatant, cells are resuspended in culture media, α-MEM (Invitrogen/Gibco) supplemented with 10% FBS (Invitrogen/Gibco) and 1% antibiotic/antimycotic (Sigma), counted using a hemocytometer and plated in T75 flasks at a density of 4,000 cells/cm2. The culture medium is changed every 2/3 days. Upon confluence cells may be trypsinized and passaged to new T75 flasks. Suspension of placental cells are subsequently washed, assessed for viability, and may either be used directly for the practice of the technology by administration either locally or systemically. Alternatively, cells may be purified for certain populations with increased biological activity. Purification may be performed using means known in the art, and described above for purification of cord blood stem cells, or may be achieved by positive selection for the following markers: SSEA3, SSEA4, TRA1-60, TRA1-81, c-kit, and Thy-1. In some situations it will be desirable to expand cells before use for generation of conditioned media. Expansion can be performed by culture ex vivo with specific growth factors [3,4].

### Bone Marrow Mononuclear Cells and Mesenchymal Cells

In one aspect of the technology, cells used for generation of conditioned media may include, for example, bone marrow mononuclear cells. Alternatively, bone marrow mesenchymal stem cells or purified hematopoietic progenitor cells may be used, for example. In another aspect of the technology, bone marrow derived CD34+ cells may be substituted with administered CD34+ cells purified from cord blood. In another aspect of the technology, bone marrow derived CD34+ cells can be substituted with administered CD34+ cells purified from, for example adipose tissue, alternatively adipose derived stromal vascular fraction cells are used for generation of conditioned media. In another aspect of the technology, bone marrow derived CD34+ cells may be substituted with administered CD133+ cells purified from bone marrow, cord blood or adipose tissue. In another aspect of the technology, embryonic stem cells are used for generation of conditioned media, said embryonic stem cells expressing one or more antigens selected from the group consisting of: stage-specific embryonic antigens (SSEA)₃, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

In another aspect of the technology, cells used for the generation of conditioned media can be a heterogenous population of bone marrow mononuclear cells. Said bone marrow stem cells may be selected, for example, based on the ability to differentiate into one or more of the following cell types: endothelial cells, smooth muscle cells, and neuronal cells. In another aspect of the technology, bone marrow stem cells are used for generation of conditioned media, said cells being selected based on expression of one or more of the following antigens: CD34, c-kit, flk-1, Stro-1, CD105, CD73, CD31, CD146, vascular endothelial-cadherin, CD133 and CXCR-4.

As described above, in some embodiments, bone marrow stem cells may be used as a cell source for generation of said conditioned media. Said bone marrow stem cells may be either used freshly isolated, freshly purified, or used subsequent to ex vivo expansion. A typical bone marrow harvest for collecting starting material for practicing one embodiment involves a bone marrow harvest with the goal of acquiring approximately 5-700 ml of bone marrow aspirate. Numerous techniques for the aspiration of marrow are described in the art and part of standard medical practice. One particular methodology that may be attractive due to decreased invasiveness is the “mini-bone marrow harvest” [8]. In one specific embodiment bone marrow mononuclear cells are isolated by pheresis or gradient centrifugation. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS3000-Plus blood-cell separator (Baxter, Deerfield, USA), the Haemonetics separator (Braintree, Mass.), or the Fresenius AS 104 and the Fresenius AS TEC 104 (Fresenius, Bad Homburg, Germany) separators. In addition to injection of mononuclear cells, purified bone marrow subpopulations may be used. Additionally, ex vivo expansion and/or selection may also be utilized for augmentation of desired biological properties for use in treatment of acne.

### Very Small Embryonic Like (VSEL) Cells

In a specific embodiment, conditioned media for use with the current technology can be generated by culture of Very Small Embryonic Like (VSEL) cells derived from bone marrow or cord blood is performed. VSEL cells comprise a population of cells that possess a small physical size (2-4 micrometers) that are capable of generating tissues from all three germ lineages [5]. VSEL are CXCR4(+), Oct-4(+) SSEA-1(+), Sca-1(+) lin(−) CD45(−) and have also been found in cord blood [6].

### Cord Blood Stem Cells

In another aspect of the technology, cord blood stem cells may be used for generation of conditioned media. Said cord blood stem cells may be purified into hematopoietic or mesenchymal lineage. Said cord blood stem cells may be multipotent and capable of differentiating into endothelial, smooth muscle, and neuronal cells. Said cord blood stem cells may be identified based on expression of one or more antigens selected from a group comprising: SSEA-3, SSEA-4, CD9, CD34, c-kit, OCT-4, Nanog, and CXCR-4 and lacking expression of one or more markers such as, for example: CD3, CD34, CD45, and CD11b.

Thus, for example, cord blood stem cells can be used as a starting population for generation of stem or progenitor conditioned media. Said cord blood stem cells may be cultured as a heterogenous population of cells commonly referred to as cord blood mononuclear cells. Said cells may be isolated according to many methods known in the art. In one particular method, cord blood is collected from fresh placenta and mononuclear cells are purified by centrifugation using a density gradient such as Ficoll or Percoll, in another method cord blood mononuclear cells are isolated from contaminating erythrocytes and granulocytes by the Hetastarch with a 6% (wt/vol) hydroxyethyl starch gradient. Cells are subsequently washed to remove contaminating debris, assessed for viability, and cultured at a concentration and time point of sufficient length to generate a conditioned media possessing anti-acne activity.

In another embodiment of the technology, cord blood stem cells can be fractionated and the fraction with enhanced ability to generate conditioned media with acne-therapeutic activity chosen. Enrichment of cells may be performed using physical differences, electrical potential differences, differences in uptake or excretion of certain compounds, as well as differences in expression marker proteins. Distinct physical property differences between stem cells with high proliferative potential and low proliferative potential are known. Accordingly, in some embodiments of the technology, it will be useful to select cord blood stem cells with a higher proliferative ability, whereas in other situations, a lower proliferative ability may be desired. In embodiments where specific cellular physical properties are the basis of differentiating between cord blood stem cells with various biological activities, discrimination on the basis of physical properties can be performed using a Fluorescent Activated Cell Sorter (FACS), through manipulation of the forward scatter and side scatter settings. Other methods of separating cells based on physical properties can include the use of filters with specific size ranges, as well as density gradients and pheresis techniques. When differentiation is desired based on electrical properties of cells, techniques such as electrophotoluminescence may be used in combination with a cell sorting means such as FACS. Selection of cells based on ability to uptake certain compounds can be performed using, for example, the ALDESORT system, which provides a fluorescent-based means of purifying cells with high aldehyde dehydrogenase activity. Cells with high levels of this enzyme are known to possess higher proliferative and self-renewal activities in comparison to cells possessing lower levels. Other methods of identifying cells with high proliferative activity includes identifying cells with ability to selectively efflux certain dyes such as rhodamine-123 and or Hoechst 33342. Without being bound to theory, cells possessing this property often express the multidrug resistance transport protein ABCG2, and are known for enhanced growth factor producing ability compared to cells which do not possess this efflux mechanism. In other embodiments, cord blood cells are purified based on expression of markers.

Certain desired activities can be endowed onto said cord blood stem cells prior to administration of conditioned media to the patient. For example, cells can be “activated” by culture in hypoxic conditions or de-differentiating agents as described herein.

### Committed Progenitor Cells

In another aspect of the technology, conditioned media can be generated by culture of a committed progenitor cell. The committed progenitor cells may be or may include, for example, one or more of: endothelial progenitor cells, neuronal progenitor cells, and hematopoietic progenitor cells. Said committed progenitor cells may be committed endothelial progenitor cells purified from the bone marrow or peripheral blood. Said committed endothelial progenitor cells may be purified from peripheral blood of a patient whose committed endothelial progenitor cells are mobilized by administration of a mobilizing agent or therapy. Said mobilizing agent may be or may include, for example, one or more of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors and small molecule antagonists of SDF-1. Said mobilization therapy may be selected from the group consisting of: exercise, hyperbaric oxygen, autohemotherapy by ex vivo ozonation of peripheral blood, and induction of SDF-1 secretion in an anatomical area outside of the bone marrow.

### Reprogrammed Cells

In another aspect of the technology, conditioned media may be generated by culture of reprogrammed stem cells. Said cells may be or may include, for example, one or more of: cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions.

In another aspect of the technology, conditioned media can be generated by culture of a reprogrammed cell, said reprogramming may be performed in vitro or in vivo with a DNA demethylating agent. The agent can be or include, for example, one or more of: 5-azacytidine, psammaplin A, and zebularine. In another aspect of the technology, conditioned media is generated by culture of a reprogrammed cell, in vitro or in vivo with a DNA histone deacetylase inhibitor, for example, one or more of: valproic acid, trichostatin-A, trapoxin A and depsipeptide.

### Endometrial Regenerative Cells

In another aspect of the technology, conditioned media can be generated by culture of endometrial regenerative cells (ERC). Said cells can be isolated from menstrual blood or alternatively directly from the endometrium and have a defined phenotype (CD90, 105 positive, CD14, CD34, Stro-1 negative). These cells have been demonstrated to possess pluripotency, capable of differentiating into heart, lung, liver, pancreas, bone, brain, fat, cartilage, and blood vessel tissue [7].

### Amniotic Fluid-Derived Stem Cells

In another aspect of the technology, conditioned media can be generated by culture of amniotic fluid-derived stem cells. Said cells may be selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1, and lack expression of one or more of the following antigens: CD34, CD45, and HLA Class II. In another aspect of the technology, conditioned media can be generated by culture of neuronal stem cells, said cells are selected based on expression of one or more of the following antigens: RC-2, 3CB2, BLB, Sox-2hh, GLAST, Pax 6, nestin, Muashi-1, NCAM, A2B5 and prominin.

Thus, in some embodiments, amniotic fluid stem cells may be utilized as a starting material for generation of conditioned media. Amniotic fluid is routinely collected during amniocentesis procedures. In one embodiment, amniotic fluid mononuclear cells can be utilized therapeutically in an unpurified manner. In other embodiments, amniotic fluid stem cells are substantially purified (for example, at least at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or at least 99.99% pure) based on expression of markers such as SSEA-3, SSEA4, Tra-1-60, Tra-1-81 and Tra-2-54, and subsequently administered. In other embodiments cells are cultured, as described in U.S. Patent Application Pub. No. 2005/0054093 (the content of which is hereby incorporated by reference in its entirety), expanded, and subsequently infused into the patient. Amniotic stem cells are described in the following references [9-11]. One particular aspect of amniotic stem cells that makes them amenable for use in practicing certain aspects of the current technology is their potent growth factor secreting and anti-inflammatory activity [12].

### Circulating Peripheral Blood Stem Cells

Alternatively, said conditioned media may be generated by culture of circulating peripheral blood stem cells. Such cells are characterized by ability to proliferate in vitro for a period of over 3 months, expression of CD34, CXCR4, CD117, CD113, and c-met, and may or may not lack substantial expression of differentiation associated markers. Said differentiation associated markers can be or include, for example, one or more of: CD2, CD3, CD4, CD11, CD11a, Mac-1, CD14, CD16, CD19, CD24, CD33, CD36, CD38, CD45, CD56, CD64, CD68, CD86, CD66b, and HLA-DR.

### Circulating Mesenchymal Stem Cells

Circulating mesenchymal stem cells may alternatively be used for generation of conditioned media, said circulating mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. Said mesenchymal stem cells do not express substantial levels of HLA-DR, CD117, and CD45. Alternatively, mesenchymal stem cells may be derived from, for example, one or more of: bone marrow, adipose tissue, umbilical cord blood, placental tissue, peripheral blood mononuclear cells, differentiated embryonic stem cells, and differentiated progenitor cells.

### Germinal Stem Cells

In another aspect of the technology, conditioned media may be generated by culture of germinal stem cells. Said germinal stem cells express markers, for example, one or more of: Oct4, Nanog, Dppa5 Rbm, cyclin A2, Tex18, Stra8, Dazl, beta1- and alpha6-integrins, Vasa, Fragilis, Nobox, c-Kit, Sca-1 and Rex1.

### Adipose Tissue Derived Stem Cells

In another aspect of the technology, conditioned media may be generated by culture of adipose tissue derived stem cells. Said adipose tissue derived stem cells express markers may be or include, for example, one or more of: CD13, CD29, CD44, CD63, CD73, CD90, CD166, Aldehyde dehydrogenase (ALDH), and ABCG2. Adipose tissue derived stem cells may be selected and/or characterized, for example, based on ability to proliferate in culture for a period of at least one month.

### Exfoliated Teeth Derived, Hair Follicle and Dermal Stem Cells

In another aspect of the technology, conditioned media may be generated by culture of exfoliated teeth derived stem cells. Said stem cells express markers may be or include, for example, one or more of: STRO-1, CD146 (MUC18), alkaline phosphatase, MEPE, and bFGF. In another aspect of the technology, conditioned media may be generated by culture of hair follicle stem cells. Said hair follicle stem cells express markers may be or include, for example, one or more of: cytokeratin 15, Nanog, and Oct-4. Additional, said hair follicle stem cells may be identified/characterized based on capability of proliferating in culture for a period of at least one month. Said hair follicle stem cells may be identified/characterized based on ability to secrete one or more of the following proteins when grown in culture: basic fibroblast growth factor (bFGF), endothelin-1 (ET-1) and stem cell factor (SCF). In another aspect of the technology, conditioned media may be generated by culture of hair dermal stem cells. Said cells express markers may be or include, for example, one or more of: CD44, CD13, CD29, CD90, and CD105. Said dermal stem cells may be capable of proliferating in culture for a period of at least one month.

### Parthenogenically Derived Stem Cells

In another aspect of the technology, conditioned media may be generated by culture of parthenogenically derived stem cells. Said cells are generated by addition of a calcium flux inducing agent to activate an oocyte followed by enrichment of cells expressing markers may be or include, for example, one or more of: SSEA-4, TRA 1-60 and TRA 1-81.

### Side Population Cells

In another aspect of the technology, conditioned media can be generated by culture of a side population cell, wherein said side population cells are identified based on expression of the multidrug resistance transport protein (ABCG2) or ability to efflux intracellular dyes such as rhodamine-123 and or Hoechst 33342. Said cells may be derived from tissues such as, for example, pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, and the like.

### Formulations and Excipients

The methods and compositions presented herein relate to treatment of acne by administering one or more, or one or more, or a mixture of components secreted by, derived from, or isolated from a stem cell or progenitor cell. It is contemplated that the components secreted by a stem cell or progenitor cell can be administered alone, or in combination with a pharmaceutically-acceptable carrier or excipient. For example, in certain aspects, the compositions can be formulated in a manner to enter the pilosebaeous gland (unit), hair follicle, epidermis, dermis, or a combination thereof. For example, the composition can be formulated in a controlled release formulation, sustained release formulation, immediate release formulation, or any combination thereof. In certain embodiments, the mixture of components can be administered in the form of an emulsion, gel, pack, cosmetic liquid or soap, ointments or patches.

Appropriate excipients for use in the present compositions may include, for example, one or more carriers, binders, fillers, vehicles, disintegrants, surfactants, dispersion or suspension aids, thickening or emulsifying agents, isotonic agents, preservatives, lubricants, and the like or combinations thereof, as suited to a particular dosage from desired. Remington: the Science and Practice of Pharmacy, Twenty-First Edition, Beringer et al. (Lippincott Williams & Wilkins, Philadelphia, Pa., 2006) discloses various formulation approaches and carriers that can be used in formulating pharmaceutically acceptable compositions and known techniques for the preparation thereof. The above-cited document is incorporated herein by reference in its entirety.

In certain embodiments, the components can further comprise a thickening agent, wherein said thickening agent is selected from the group consisting of cetostearyl alcohol, hydrogenated lanolin, aluminum stearate, propylene glycol, and polyethylene glycol.

In certain aspects, the components further comprise a chelating agent, wherein said chelating agent: (a) is present in an amount of about 0.0005% to about 1.0%: (b) is selected from the group consisting of ethylenediamine, ethylenediaminetetraacetic acid, and dimercaprol; or (c) any combination thereof.

In certain aspects, the components further comprise an emulsifying agent, said emulsifying agent comprising: (a) an aqueous phase; (b) about 1% oil to about 80% oil; (c) about 0.1% organic solvent to about 50% organic solvent; (d) at least one surfactant present in an amount of about 0.001% surfactant to about 10% surfactant; (e) about 0.0005% to about 1.0% of a chelating agent; or (f) any combination thereof.

In certain aspects, the emulsifying agent comprises: (a) an aqueous phase; (b) about 5% oil to about 80% oil; (c) about 0.1% organic solvent to about 10% organic solvent; (d) at least one non-ionic surfactant present in an amount of about 0.1% to about 10%; (e) at least one cationic agent present in an amount of about 0.01% to about 2%; (f) about 0.0005% to about 1.0% of a chelating agent; or (g) any combination thereof.

In certain aspects, the components further may include an organic solvent, said organic solvent selected from the group consisting of C₁-C₁₂ alcohol, diol, triol, dialkyl phosphate, tri-alkyl phosphate, semi-synthetic derivatives thereof, and combinations thereof; said organic solvent is an alcohol which is selected from the group consisting of a nonpolar solvent, a polar solvent, a protic solvent, and an aprotic solvent; said organic solvent is selected from the group consisting of ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof;

In certain aspects, the components further may include an oil, wherein said oil comprises: (a) any cosmetically or pharmaceutically acceptable oil; (b) a non-volatile oil; (c) an oil selected from the group consisting of animal oil, vegetable oil, natural oil, synthetic oil, hydrocarbon oils, silicone oils, and semi-synthetic derivatives thereof; (d) an oil selected from the group consisting of mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranilate, Cetyl octanoate, Octyl salicylate, Isopropyl myristate, neopentyl glycol dicarpate cetols, Ceraphyls®, Decyl oleate, diisopropyl adipate, C C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and combinations thereof; or (e) any combination thereof.

In certain aspects, the components may further include a silicone component, wherein said silicone compound comprises at least one volatile silicone oil, wherein: (a) the volatile silicone oil can be the sole oil in the silicone component or is combined with other silicone and non-silicone oils, and wherein the other oils can be volatile or non-volatile; (b) the volatile oil used in the silicone component is different than the oil in the oil phase; (c) the silicone component may be or include, for example, one or more of methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof; or (d) any combination thereof.

In certain aspects, the components may be admixed with a volatile oil, wherein: (a) the volatile oil can be the organic solvent, or the volatile oil can be present in addition to an organic solvent; (b) the volatile oil is a terpene, monoterpene, sesquiterpene, carminative, azulene, semi-synthetic derivatives thereof, or combinations thereof; (c) the volatile oil may be or include, for example, one or more of a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives thereof, and combinations thereof; or (d) any combination thereof.

In some embodiments, the components can further comprise a surfactant. The surfactant can be any suitable surfactant for the methods and compositions provided herein. In some embodiments, the surfactant can be a pharmaceutically acceptable ionic surfactant, a pharmaceutically acceptable nonionic surfactant, a pharmaceutically acceptable cationic surfactant, a pharmaceutically acceptable ionic surfactant, a pharmaceutically acceptable anionic surfactant, or a pharmaceutically acceptable zwitterionic surfactant;

In some embodiments, the surfactant can be, for example, a pharmaceutically acceptable ionic polymeric surfactant, a pharmaceutically acceptable nonionic polymeric surfactant, a pharmaceutically acceptable cationic polymeric surfactant, a pharmaceutically acceptable anionic polymeric surfactant, or a pharmaceutically acceptable zwitterionic polymeric surfactant.

In some embodiments, the surfactant can be, for example, a polymeric surfactant which may be or include, for example, one or more of a graft copolymer of a poly(methyl methacrylate) backbone with at least one polyethylene oxide (PEO) side chain, polyhydroxystearic acid, an alkoxylated alkyl phenol formaldehyde condensate, a polyalkylene glycol modified polyester with fatty acid hydrophobes, a polyester, semi-synthetic derivatives thereof, and combinations thereof.

In some embodiments, the surfactant can be, for example, selected from the group consisting of ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxide and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxystearate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thighlycolate, Glyceryl dilaurate, Glyceryl dioleate, Glyceryl dimyristate, Glyceryl distearate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isopropyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, and mixtures thereof.

In some embodiments, the surfactant can be, for example, a non-ionic lipid selected from the group consisting of glyceryl laurate, glyceryl myristate, glyceryl dilaurate, glyceryl dimyristate, semi-synthetic derivatives thereof, and mixtures thereof.

In some embodiments, the surfactant can be, for example, a polyoxyethylene fatty ether having a polyoxyethylene head group ranging from about 2 to about 100 groups.

In some embodiments, the surfactant can be, for example, an alkoxylated alcohol, for example, an ethoxylated derivative of lanolin alcohol.

In some embodiments, the surfactant can be, for example, nonionic and may be or include, for example, one or more of nonoxynol-9, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, bis(polyethylene glycol bis[imidazoyl carbonyl]), Brij® 35, Brij® 56, Brij® 72, Brij® 76, Brij® 92V, Brij® 97, Brij® 58P, Cremophor® EL, decaethylene glycol monododecyl ether, N-Decanoyl-N-methylglucamine, n-Decyl alpha-D-glucopyranoside, decyl beta-D-maltopyranoside, n-Dodecanoyl-N-methylglucamide, n-Dodecyl alpha-D-maltoside, n-Dodecyl beta-D-maltoside, heptaethylene glycol monodecyl ether, heptaethylene glycol monotetradecyl ether, heptaethylene glycol monododecyl ether, n-Hexadecyl beta-D-maltoside, hexaethylene glycol monododecyl ether, hexaethylene glycol monohexadecyl ether, hexaethylene glycol monooctadecyl ether, hexaethylene glycol monotetradecyl ether, igepal CA-630, Methyl-6-O—(N-heptylcarbamoyl)-alpha-D-glucopyranoside, nonaethylene glycol monododecyl ether, n-Nonanoyl-N-methylglucamine, octaethylene glycol monodecyl ether, octaethylene glycol monododecyl ether, octaethylene glycol monohexadecyl ether, octaethylene glycol monooctadecyl ether, octaethylene glycol monotetradecyl ether, octyl-beta-D-glucopyranoside, pentaethylene glycol monodecyl ether, pentaethylene glycol monododecyl ether, pentaethylene glycol monohexadecyl ether, pentaethylene glycol monohexyl ether, pentaethylene glycol monooctadecyl ether, pentaethylene glycol monooctyl ether, polyethylene glycol diglycidyl ether, polyethylene glycol ether W-1, polyoxyethylene 10 tridecyl ether, polyoxyethylene 100 stearate, polyoxyethylene 20 isohexadecyl ether, polyoxyethylene 20 oleyl ether, polyoxyethylene 40 stearate, polyoxyethylene 50 stearate, polyoxyethylene 8 stearate, polyoxyethylene bis(imidazolyl carbonyl), polyoxyethylene 25 propylene glycol stearate, saponin from Quillaja bark, Span® 20, Span® 40, Span® 60, Span® 65, Span® 80, Span® 85, Tergitol, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton X-114, Triton X-165, Triton X-305, Triton X-405, Triton X-45, Triton X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapoln-Undecyl beta-D-glucopyranoside, poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 Benzoate, poloxamer 182 Dibenzoate, semi-synthetic derivatives thereof, and combinations thereof.

In some embodiments, the surfactant can be, for example, cationic and may be or include, for example, one or more of a quarternary ammonium compound, an alkyl trimethyl ammonium chloride compound, a dialkyl dimethyl ammonium chloride compound, Benzalkonium chloride, Benzyldimethylhexadecylammonium chloride, Benzyldimethyltetradecylammonium chloride, Benzyldodecyldimethylammonium bromide, Benzyltrimethylammonium tetrachloroiodate, cetylpyridinium chloride, dimethyldioctadecylammonium bromide, dodecylethyldimethylammonium bromide, dodecyltrimethylammonium bromide, ethylhexadecyldimethylammonium bromide, Girard's reagent T, hexadecyltrimethylammonium bromide, n,n′,n′-Polyoxyethylene(10)-N-tallow-1,3-diaminopropane, Thonzonium bromide, Trimethyl(tetradecyl)ammonium bromide, 1,3,5-Triazine-1,3,5(2H,4H,6H)-triethanol, 1-Decanaminium, N-decyl-N,n-dimethyl-, chloride, didecyl dimethyl ammonium chloride, 2-(2-(p-(Diisobutyl)cresosxy)ethoxy)ethyl dimethyl benzyl ammonium chloride, 2-(2-(p-(Diisobutyl)phenoxy)ethoxy)ethyl dimethyl benzyl ammonium chloride, alkyl 1 or 3 benzyl-1-(2-hydroxyethyl)-2-imidazolinium chloride, alkyl bis(2-hydroxyethyl)benzyl ammonium chloride, alkyl demethyl benzyl ammonium chloride, alkyl dimethyl 3,4-dichlorobenzyl ammonium chloride (100% C12), alkyl dimethyl 3,4-dichlorobenzyl ammonium chloride (50% C14, 40% C12, 10% C16), alkyl dimethyl 3,4-dichlorobenzyl ammonium chloride (55% C14, 23% C12, 20% C16), alkyl dimethyl benzyl ammonium chloride, alkyl dimethyl benzyl ammonium chloride (100% C14), alkyl dimethyl benzyl ammonium chloride (100% C16), alkyl dimethyl benzyl ammonium chloride (41% C14, 28% C12), alkyl dimethyl benzyl ammonium chloride (47% C12, 18% C14), alkyl dimethyl benzyl ammonium chloride (55% C16, 20% C14), alkyl dimethyl benzyl ammonium chloride (58% C14, 28% C16), alkyl dimethyl benzyl ammonium chloride (60% C14, 25% C12), alkyl dimethyl benzyl ammonium chloride (61% C11, 23% C14), alkyl dimethyl benzyl ammonium chloride (61% C12, 23% C14), alkyl dimethyl benzyl ammonium chloride (65% C12, 25% C14), alkyl dimethyl benzyl ammonium chloride (67% C12, 24% C14), alkyl dimethyl benzyl ammonium chloride (67% C12, 25% C14), alkyl dimethyl benzyl ammonium chloride (90% C14, 5% C12), alkyl dimethyl benzyl ammonium chloride (93% C14, 4% C12), alkyl dimethyl benzyl ammonium chloride (95% C16, 5% C18), alkyl didecyl dimethyl ammonium chloride, alkyl dimethyl benzyl ammonium chloride (C12-16), alkyl dimethyl benzyl ammonium chloride (C12-18), dialkyl dimethyl benzyl ammonium chloride, alkyl dimethyl dimethylbenzyl ammonium chloride, alkyl dimethyl ethyl ammonium bromide (90% C14, 5% C16, 5% C12), alkyl dimethyl ethyl ammonium bromide (mixed alkyl and alkenyl groups as in the fatty acids of soybean oil), alkyl dimethyl ethylbenzyl ammonium chloride, alkyl dimethyl ethylbenzyl ammonium chloride (60% C14), alkyl dimethyl isopropylbenzyl ammonium chloride (50% C12, 30% C14, 17% C16, 3% C18), alkyl trimethyl ammonium chloride (58% C18, 40% C16, 1% C14, 1% C12), alkyl trimethyl ammonium chloride (90% C18, 10% C16), alkyldimethyl(ethylbenzyl) ammonium chloride (C12-18), di-(C8-10)-alkyl dimethyl ammonium chlorides, dialkyl dimethyl ammonium chloride, dialkyl methyl benzyl ammonium chloride, didecyl dimethyl ammonium chloride, diisodecyl dimethyl ammonium chloride, dioctyl dimethyl ammonium chloride, dodecyl bis(2-hydroxyethyl) octyl hydrogen ammonium chloride, dodecyl dimethyl benzyl ammonium chloride, dodecylcarbamoyl methyl dimethyl benzyl ammonium chloride, heptadecyl hydroxyethylimidazolinium chloride, hexahydro-1,3,5-tris(2-hydroxyethyl)-s-triazine, Myristalkonium chloride (and) Quat RNIUM 14, n,n-Dimethyl-2-hydroxypropylammonium chloride polymer, n-Tetradecyl dimethyl benzyl ammonium chloride monohydrate, octyl decyl dimethyl ammonium chloride, octyl dodecyl dimethyl ammonium chloride, octyphenoxyethoxyethyl dimethyl benzyl ammonium chloride, oxydiethylenebis(alkyl dimethyl ammonium chloride), Trimethoxysily propyl dimethyl octadecyl ammonium chloride, Trimethoxysilyl quats, Trimethyl dodecylbenzyl ammonium chloride, semi-synthetic derivatives thereof, and combinations thereof.

In some embodiments, the surfactant can be, for example, anionic and may be or include, for example, one or more of a carboxylate, a sulphate, a sulphonate, a phosphate, chenodeoxycholic acid, chenodeoxycholic acid sodium salt, cholic acid, ox or sheep bile, dehydrocholic acid, deoxycholic acid, deoxycholic acid methyl ester, digitonin, digitoxigenin, n,n-Dimethyldodecylamine N-oxide, docusate sodium salt, glycochenodeoxycholic acid sodium salt, glycocholic acid hydrate, synthetic, glycocholic acid sodium salt hydrate, synthetic, glycodeoxycholic acid monohydrate, glycodeoxycholic acid sodium salt, glycolithocholic acid 3-sulfate disodium salt, glycolithocholic acid ethyl ester, n-Lauroylsarcosine sodium salt, n-Lauroylsarcosine solution, lithium dodecyl sulfate, lugol solution, niaproof 4, Type 4,1-Octanesulfonic acid sodium salt, sodium 1-butanesulfonate, sodium 1-decanesulfonate, sodium 1-dodecanesulfonate, sodium 1-heptanesulfonate anhydrous, sodium 1-nonanesulfonate, sodium 1-propanesulfonate monohydrate, sodium 2-bromoethanesulfonate, sodium cholate hydrate, sodium choleate, sodium deoxycholate, sodium deoxycholate monohydrate, sodium dodecyl sulfate, sodium hexanesulfonate anhydrous, sodium octyl sulfate, sodium pentanesulfonate anhydrous, sodium taurocholate, taurochenodeoxycholic acid sodium salt, taurodeoxycholic acid sodium salt monohydrate, taurohyodeoxycholic acid sodium salt hydrate, taurolithocholic acid 3-sulfate disodium salt, tauroursodeoxycholic acid sodium salt, Trizma® dodecyl sulfate, ursodeoxycholic acid, semi-synthetic derivatives thereof, and combinations thereof;

In some embodiments, the surfactant can be, for example, zwitterionic and may be or include, for example, one or more of an N-alkyl betaine, lauryl amindo propyl dimethyl betaine, an alkyl dimethyl glycinate, an N-alkyl amino propionate, cHAPS, minimum 98%, cHAPS, minimum 98%, cHAPS, for electrophoresis, minimum 98%, cHAPSO, minimum 98%, cHAPSO, cHAPSO, for electrophoresis, 3-(Decyldimethylammonio)propanesulfonate inner salt, 3-(Dodecyldimethylammonio)propanesulfonate inner salt, 3-(N,n-Dimethylmyristylammonio)propanesulfonate inner salt, 3-(N,n-Dimethyloctadecylammonio)propanesulfonate, 3-(N,n-Dimethyloctylammonio)propanesulfonate inner salt, 3-(N,n-Dimethylpalmitylammonio)propanesulfonate, semi-synthetic derivatives thereof, and combinations thereof.

In certain embodiments where the surfactant is an alkoxylated alcohol, the surfactant can be the ethoxylated derivative of lanolin alcohol, for example, laneth-10, which is the polyethylene glycol ether of lanolin alcohol with an average ethoxylation value of 10.

In some embodiments, the mixture of components is admixed with: (a) at least one preservative; (b) at least one pH adjuster; (c) at least pharmaceutically acceptable buffer; or (d) any combination thereof.

For example, in some embodiments, the preservative may be or include, for example, one or more of cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic Acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof.

In some embodiments, the pH adjuster can be any suitable agent to maintain the desired pH. For example, the pH adjuster can be a pH adjuster which may be or include, for example, one or more of diethanolamine, lactic acid, monoethanolamine, triethylanolamine, sodium hydroxide, sodium phosphate, semi-synthetic derivatives thereof, and combinations thereof.

The buffer can be any suitable buffer. In some embodiments, the buffer can be or include, for example, one or more of 2-Amino-2-methyl-1,3-propanediol, 2-Amino-2-methyl-1-propanol, L-(+)-Tartaric acid, ACES, ADA, acetic acid, ammonium acetate, ammonium bicarbonate, ammonium citrate dibasic, formate solution, ammonium formate, ammonium oxalate monohydrate, ammonium phosphate dibasic solution, ammonium phosphate dibasic, ammonium phosphate monobasic solution, ammonium phosphate monobasic, ammonium sodium phosphate dibasic tetrahydrate, ammonium sulfate solution, ammonium tartrate dibasic solution, ammonium tartrate dibasic, BES buffered saline, BES, BICINE, Bicarbonate buffer solution, NaH CO₃, Boric acid, cAPS, cHES, calcium acetate hydrate, calcium carbonate, precipitated, calcium citrate tribasic tetrahydrate, citrate Concentrated Solution, citric acid, diethanolamine, EPPS, ethylenediaminetetraacetic acid disodium salt dihydrate, formic acid solution, Gly-Gly-Gly, Gly-Gly, Glycine, HEPES, imidazole, Lipoprotein Refolding Buffer, lithium acetate dihydrate, lithium citrate tribasic tetrahydrate, MES hydrate, MES monohydrate, MES solution, MOPS, Magnesium acetate solution, Magnesium acetate tetrahydrateMagnesium citrate tribasic nonahydrate, Magnesium formate solution, Magnesium phosphate dibasic trihydrate, oxalic acid dihydrate, pIPES, phosphate buffered saline, piperazine, potassium D-tartrate monobasic, potassium acetate solution, potassium bicarbonate, potassium chloride, potassium citrate monobasic, potassium citrate tribasic solution, potassium formate, potassium oxalate monohydrate, potassium phosphate dibasic, potassium phosphate monobasic, potassium phosphate tribasic monohydrate, potassium phthalate monobasic, potassium sodium tartrate tetrahydrate, potassium tetroxalate dihydrate, propionic acid solution, sTE buffer solution, sTET buffer solution, sodium 5,5-diethylbarbiturate, sodium acetate solution, sodium acetate trihydrate, sodium bicarbonate, sodium bitartrate monohydrate, sodium carbonate decahydrate, sodium carbonate, anhydrous sodium citrate monobasic, sodium citrate tribasic dihydrate, sodium formate solution, sodium oxalate, sodium phosphate dibasic dihydrate, sodium phosphate dibasic dodecahydrate, sodium phosphate monobasic dihydrate, sodium phosphate monobasic solution, sodium pyrophosphate dibasic, sodium pyrophosphate tetrabasic decahydrate, sodium tartrate dibasic dihydrate, sodium tartrate dibasic solution, 1sodium tetraborate decahydrate, TAPS, TES, TM buffer solution, TNT buffer solution, TRIS Glycine buffer solution, TRIS acetate-EDTA buffer solution, TRIS buffered saline, TRIS glycine SDS buffer solution, TRIS phosphate-EDTA buffer solution, Tricine, Triethanolamine, Triethylamine, Triethylammonium acetate buffer, Triethylammonium phosphate solution, Trimethylammonium acetate solution, Trimethylammonium phosphate solution, Tris-EDTA buffer solution, Trizma® acetate, Trizma® base, Trizma® carbonate, Trizma® hydrochloride buffer solution, Trizma® maleate.

In certain aspects, the mixture of components is formulated in a manner to enter the pilosebaeous gland (unit), hair follicle, epidermis, dermis, or a combination thereof. In certain aspects, the mixture of components is formulated in a controlled release formulation, sustained release formulation, immediate release formulation, or any combination thereof.

In certain aspects, the mixture of components is formulated with an excipient which may be or include, for example, one or more of: carbomer 940, dimethicone, disodium lauryl sulfosuccinate, edentate disodium, glycerin, hydrated silica, methylparaben, poloxamer 182, sodium hydroxide, phosphate buffered saline, water, polymers of polyvinyl chloride, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl flouride, polyvinyl imidazole, chlorosulphonated polyolefins, and polyvinyl alcohol.

Having now generally described the technology, it will be more readily understood through reference to the following examples, which are provided by way of illustration and are not intended to be limiting in regard to the scope of the invention, unless specified.

### EXAMPLES

### Example 1

### Generation of Stem Cell Conditioned Media

Placental mesenchymal stem cells were derived by removing the epithelium of the umbilical cord section along its length to expose the underlying Warton's Jelly. Each vessel, with its surrounding Wharton's Jelly matrix, was then pulled away, after which the ends of each dissected vessel were tied together with a suture creating “loops” that were placed into a 50-mL tube containing a solution of 0.5-0.75 mg/mL collagenase (Sigma, St. Louis, Mo.) with phosphate-buffered saline (PBS, Invitrogen/Gibco, Carlsbad, Calif.). After 18 h, the loops were removed from the suspension, which was then diluted with PBS to reduce the viscosity of the suspension and centrifuged. Following the removal of the supernatant, cells were resuspended in culture media, α-MEM (Invitrogen/Gibco) supplemented with 10% FBS (Invitrogen/Gibco) and 1% antibiotic/antimycotic (Sigma), counted using a hemocytometer and plated in T75 flasks at a density of 4,000 cells/cm2. The culture medium was changed every 2/3 days. Upon confluence cells were trypsinized and passaged to new T75 flasks. Suspension of placental cells was subsequently washed, and cultured in RPMI media without phenol red or FBS for 24 hours. The media was collected and sterile filtered to exclude cells and cellular debris. The media was then mixed at a 1:1 volume ratio with a moisturizing cream.

### Example 2

### TREATMENT OF PATIENT WITH STEM CELL CONDITIONED MEDIA

A 15-year-old female with a 2-year history of severe acne was seen. She was using a facial wash containing 5% benzyl peroxide daily and orally taking 100 mg of doxycycline daily. In spite of those treatments she presented with 22 inflamed popular acne lesions, and 6 non-inflammatory popular lesions on her face. She applied the media/moisturizing cream described in Example 1 to her face for 10 days. After 3 days 18 of the inflammatory lesions and 1 of the non-inflammatory lesions had resolved. The redness of her face had considerably decreased. At the end of 10 days only 2 non-inflammatory lesions remained on her face.

### Example 3

### Clinical Trial Evaluation of Mesenchymal Stem Cell Condition Media for Treatment of Acne-Vulgaris

An open-label clinical trial is conducted in 25 patients with a mean age of 21.0.+−.4.18 years with 9 of male and 16 female patients after obtaining their informed consent.

Patients in the trial are selected for based on the inclusion/exclusion criteria in the table below:

Inclusion Criteria:
Males and females between 12 to 25 years of age
Presence of inflammatory and non-inflammatory
lesions At least 10 inflammatory lesions with
maximum 3 nodules and pseudocysts
Able to follow-up according to protocol
Patients not taking any medication for treatment
of Acne in preceding one month.
Exclusion Criteria:
Acne Conglobata
Pregnant or Breast feeding females or females
having intention of becoming pregnant
Significant systemic disease
Any drug/ alcohol addiction
History of chronic diseases treated with
medications in the preceding month which might
affect acne condition and treatment outcome

The patients are requested to use media/moisturizing cream (Conditioned media) as described in Example 1. Conditioned media is administered topically twice a day after washing the face with a mild soap. Administration is performed over the whole face and conditioned media is not washed off after application.

The patients are evaluated on Day 14 and on Day-28 of the study for black heads, inflamed papules, inflamed pustules, cysts, nodules, white heads and blemishes. The parameters are reviewed at initial and at the end of 14 and 28 days. Apart from above parameters the stem cell conditioned media is also evaluated for the cosmetic effect such as; exfoliation, moisturizing effect, smoothening effect, soothing effect and healing without scar formation etc.

The patients and the investigators are asked to rate the outcome of the treatment separately and the severity score was recorded and analyzed on all the days. The local/systemic adverse effects, if any, are noted during follow-up of the study.

Results are presented below as a comparison to pre-treatment values

| | Pre-treatment | Day 14 | Day 28 |
|---|---|---|---|
| 1. Open Comedones | 100% | 54% | 8% |
| 2. Closed Comedones | 100% | 12% | 11% |
| 3. Inflamed Papules | 100% | 74% | 5% |
| 4. Pustules | 100% | 10% | 4% |
| 5. Blemishes | 100% | 45% | 5% |
| 6. Erythema | 100% | 5% | 12% |

### Example 4

Prevention of Acne-Vulgaris

A 16-year-old male with a 2-year history of episodic acne is seen. The patient is currently not suffering from any major lesions. He applies the media/moisturizing cream described in Example 1 to the left side of his face for 90 days. During that same time period, he also applies a moisturizing cream lacking the conditioned media to the right side of his face.

The patient's acne condition is evaluated every 30 days to monitor acne flare ups. After 60 days, the right side of the patient's face has 10 inflamed popular acne lesions and 3 non-inflammatory popular lesions. In contrast, the left side of the patient's face is clear of lesions.

### Example 5

### Prevention of Scarring Caused by Acne-Vulgaris

An 18-year-old male with a 4-year history of severe acne is seen. The patient suffers from scarring from previous acne outbreaks. He applies the media/moisturizing cream described in Example 1 to the left side of his face for six months. During that same time period, he also applies a moisturizing cream lacking the conditioned media to the right side of his face.

The patient's acne condition is evaluated after six months to monitor scarring. The right side of the patient's face has increased scar tissue caused by acne. In contrast, the left side of the patient's face has significantly fewer new scar tissues.

### Example 6

### Ameliorating Scarring Caused by Acne-Vulgaris

A 17-year-old female with a 2-year history of severe acne is seen. The patient suffers from scarring from previous acne outbreaks. She applies the media/moisturizing cream described in Example 1 to the left side of her face for 90 days. During that same time period, she also applies a moisturizing cream lacking the conditioned media to the right side of her face.

The patient is evaluated after 90 days to monitor scarring. The right side of the patient's face has no change in scar tissue caused by acne. In contrast, scarring on the left side of the patient's face has decreased compared to 90 days earlier.

### REFERENCES

[1] R. Demir, G. Kosanke, G. Kohnen, S. Kertschanska and P. Kaufmann Classification of human placental stem villi: review of structural and functional aspects, Microsc Res Tech 38 (1997) 29-41.
[2] C. B. Portmann-Lanz, A. Schoeberlein, A. Huber, R. Sager, A. Malek, W. Holzgreve and D. V. Surbek Placental mesenchymal stem cells as potential autologous graft for pre- and perinatal neuroregeneration, Am J Obstet Gynecol 194 (2006) 664-673.
[3] A. A. Mohamed, A. M. Ibrahim, M. W. El-Masry, I. M. Mansour, M. A. Khroshied, H. M. Gouda and R. M. Riad Ex vivo expansion of stem cells: defining optimum conditions using various cytokines, Lab Hematol 12 (2006) 86-93.
[4] I. Kashiwakura and T. A. Takahashi Fibroblast growth factor and ex vivo expansion of hematopoietic progenitor cells, Leuk Lymphoma 46 (2005) 329-333.
[5] M. Kucia, R. Reca, F. R. Campbell, E. Zuba-Surma, M. Majka, J. Ratajczak and M. Z. Ratajczak A population of very small embryonic-like (VSEL) CXCR4(+)SSEA-1(+)Oct-4+ stem cells identified in adult bone marrow, Leukemia 20 (2006) 857-869.
[6] M. Kucia, M. Halasa, M. Wysoczynski, M. Baskiewicz-Masiuk, S. Moldenhawer, E. Zuba-Surma, R. Czajka, W. Wojakowski, B. Machalinski and M. Z. Ratajczak Morphological and molecular characterization of novel population of CXCR4+ SSEA-4+ Oct-4+ very small embryonic-like cells purified from human cord blood: preliminary report, Leukemia 21 (2007) 297-303.
[7] X. Meng, T. E. Ichim, J. Zhong, A. Rogers, Z. Yin, J. Jackson, H. Wang, W. Ge, V. Bogin, K. W. Chan, B. Thebaud and N. H. Riordan Endometrial regenerative cells: a novel stem cell population, J Transl Med 5 (2007) 57.
[8] S. Mineishi [Immunobiology of mini-transplant], Nippon Rinsho 61 (2003) 1489-1494.
[9] P. Bossolasco, T. Montemurro, L. Cova, S. Zangrossi, C. Calzarossa, S. Buiatiotis, D. Soligo, S. Bosari, V. Silani, G. L. Deliliers, P. Rebulla and L. Lazzari Molecular and phenotypic characterization of human amniotic fluid cells and their differentiation potential, Cell Res 16 (2006) 329-336.
[10] S. Sartore, M. Lenzi, A. Angelini, A. Chiavegato, L. Gasparotto, P. De Coppi, R. Bianco and G. Gerosa Amniotic mesenchymal cells autotransplanted in a porcine model of cardiac ischemia do not differentiate to cardiogenic phenotypes, Eur J Cardiothorac Surg 28 (2005) 677-684.
[11] A. R. Prusa, E. Marton, M. Rosner, D. Bettelheim, G. Lubec, A. Pollack, G. Bernaschek and M. Hengstschlager Neurogenic cells in human amniotic fluid, Am J Obstet Gynecol 191 (2004) 309-314.
[12] M. S. Tsai, S. M. Hwang, Y. L. Tsai, F. C. Cheng, J. L. Lee and Y. J. Chang Clonal amniotic fluid-derived stem cells express characteristics of both mesenchymal and neural stem cells, Biol Reprod 74 (2006) 545-551.

The above-cited references are incorporated by reference in their entireties.

What is claimed is:

1. A method for the treatment of acne comprising
selecting a patient in need of acne treatment;
administering to said patient a mixture of components secreted by a stem or progenitor cell.

2. The method of claim 1, wherein said mixture of components secreted by a stem or progenitor cell comprises a supernatant of a cultured cell population.

3. The method of claim 2, wherein said supernatant is obtained by culturing viable stem or progenitor cells under conditions that are physiological or near-physiological.

4. The method of claim 2, wherein said supernatant is obtained by culturing viable stem or progenitor cells under conditions that are non-physiological.

5. The method of claim 2, wherein said supernatant of a cultured cell population is substantially free of cellular debris.

6. The method of claim 2, wherein said cultured cells are exposed to conditions selected from the group consisting of: a) exposure to hypoxia; b) treatment with a histone deacetylase inhibitor; c) treatment with a growth factor; d) treatment with a DNA methyltransferase inhibitor; and e) exposure to hyperthermia.

7. The method of claim 1, wherein said stem cell is totipotent, capable of differentiating into cells of all histological types of the body.

8. The method of claim 1, wherein said at least one stem cell is pluripotent, capable of differentiating into numerous cells of the body.

9. The method of claim 1, wherein said at least one stem cell is a progenitor cell, capable of differentiating into a restricted tissue type.

10. The method of claim 7, wherein said totipotent stem cell is selected from the group consisting of: an embryonic stem cell, an extra-embryonic stem cell, a cloned stem cell, and a parthenogenesis derived cell.

11. The method of claim 8, wherein said pluripotent stem cell is selected from the group consisting of a hematopoietic stem cell, an adipose stem cell, a mesenchymal stem cell, a cord blood stem cell, a placental stem cell, an exfoliated tooth derived stem cell, an endometrial regenerative cell, a hair follicle stem cell and a neural stem cell.

12. The method of claim 9, wherein said progenitor stem cell is selected from the group consisting of neuronal, hepatic, nephrogenic, adipogenic, osteoblastic, osteoclastic, alveolar, cardiac, intestinal, and endothelial progenitor cells.

13. The method of claim 1 wherein said mixture of components is administered in the form of an emulsion, gel, pack, cosmetic liquid or soap, ointments or patches.

14. The method of claim 1, wherein said acne is a condition selected from the group consisting of acne venenata, acne vulgaris, cystic acne, acne atrophica, acne conglobata, bromide acne, chlorine acne, acne cosmetica, acne detergicans, epidemic acne, acne estivalis, acne fulminans, halogen acne, acne indurata, iodide acne, acne keloid, acne mechanica; acne papulosa, pomade acne, premenstral acne, acne pustulosa, acne rosacea, acne scorbutica, acne scrofulosorum, acne urticata, acne varioliformis, propionic acne, acne excoriee, gram negative acne, steroid acne, and nodulocystic acne.

15. The method of claim 1, wherein said mixture of components is formulated in a manner to enter the pilosebaeous gland (unit), hair follicle, epidermis, dermis, or a combination thereof.

16. The method of claim 1, wherein said mixture of components is formulated in a controlled release formulation, sustained release formulation, immediate release formulation, or any combination thereof.

17. The method of claim 1, wherein said mixture of components is admixed with at least one anti-acne agent.

18. The method of claim 17, wherein the anti-acne agent is selected from the group consisting of benzoyl peroxide, salicylic acid and a retinoid.

19. The method of claim 1, wherein said administering comprises topical administration.

20. A composition for treatment of acne comprising:
a mixture of components secreted by a stem or progenitor cell; and
a pharmaceutically-acceptable carrier or excipient.

21. The composition of claim 20, wherein said composition is formulated in a manner to enter the pilosebaeous gland (unit), hair follicle, epidermis, dermis, or a combination thereof.

22. The composition of claim 20, wherein said composition is formulated in a controlled release formulation, sustained release formulation, immediate release formulation, or any combination thereof.

23. The composition of claim 20, wherein said composition is admixed with at least one anti-acne agent.

24. The composition of claim 23, wherein the anti-acne agent is selected from the group consisting of benzoyl peroxide, salicylic acid and a retinoid.

25. A method for the prevention of acne comprising
selecting a patient in need of acne prevention;
administering to said patient a mixture of components secreted by a stem or progenitor cell.

26. A method for the prevention of scarring associated with acne comprising
selecting a patient in need of prevention of scarring associated with acne;
administering to said patient a mixture of components secreted by a stem or progenitor cell.

27. A method for ameliorating scarring associated with acne comprising
selecting a patient in need of amelioration of scarring associated with acne;
administering to said patient a mixture of components secreted by a stem or progenitor cell.
